(19) United States
(12) Patent Application Publication
(10) Pub. No.: US 2023/0070862 A1
(43) Pub. Date: Mar. 9, 2023

(54) Systems and method for viral detection

(71) Applicants: Joshua Paul Steimel, Stockton, CA (US); Alfredo Alexander-Katz, Cambridge, MA (US)

(72) Inventors: Joshua Paul Steimel, Stockton, CA (US); Alfredo Alexander-Katz, Cambridge, MA (US)

(73) Assignee: Tribosense Technologies LLC, Stockton, CA (US)

(21) Appl. No.: 17/471,105

(22) Filed: Sep. 9, 2021

(51) Int. Cl. G01N 33/543; G01N 33/569; G01N 33/58

(52) U.S. Cl. CPC G01N 33/54326; G01N 33/56983; G01N 33/582

(57) ABSTRACT

Systems and methods are provided for detecting viral particles, viral proteins, viral RNA, or viral DNA in multiple fluids. The methods consist of applying a magnetic torque to functionalized magnetic beads in a fluid solution resting on a functionalized substrate. The solution is comprised of one of the following: intact viral particles, viral proteins, RNA, or DNA. The presence and/or quantity of the aforementioned molecules or viruses is detected by measuring the translational velocity of the beads. The methods here described can detect multiple different species simultaneously using a multiplexed assay. Also, the systems here included are able to process multiple samples simultaneously.

### CROSS REFERENCES TO RELATED APPLICATIONS

This application claims the benefit of Applicants' prior provisional application, No. 63/076,377, filed on [Sep. 9, 2020].

### FIELD OF INVENTION

The technology relates to the general field of [bioassay platforms], and has certain specific application to [detecting viral antigens or particles].

### BACKGROUND

Viral infections constitute one of the biggest threats to human health. A prominent example of this is the SARS-CoV-2 pandemic that originated in China and has spread all over the world, killing almost 1 million people in less than a year. The spread of these pandemics can be controlled by having rapid, robust, cheap, and accurate tests that can assist in identifying and then isolating infected individuals in a population. Currently, there are 2 different types of tests to detect the presence of viruses in different fluids: molecular tests and antigen tests. In both cases, the read-out of current tests are colorimetric or fluorescence based, meaning one gets a light signal and converts this to a positive or negative result, and in some cases to a given concentration of the analyte being measured. An example of molecular tests PCR where RNA is converted to DNA and then amplified. PCR is slow, expensive, and requires laboratory equipment and clean rooms. Efforts have been made in making such systems small and portable. Currently, there are a few examples of point-of-care devices using similar methods. On the other hand there are antigen tests such as lateral flow assays (LFAs) or enzyme-linked immunosorbent assays (ELISAs). LFAs, like the ones you find for pregnancy tests, are cheap, but they lack the sensitivity of molecular diagnostics, despite their specificity being particularly high. Furthermore, they do not provide quantitative results regarding the amount of virus unless a lengthy process of quantitative ELISA is undertaken.

Although these two methods of detecting viral infections are well established, it would be desirable to have a quick and sensitive platform for the quantitative detection of viral particles, viral proteins, viral RNA, or viral DNA. Furthermore, being able to detect multiple viruses at the same time would be beneficial in times when there are several infections present in the population. Such an ability is called multiplexing. Being able to detect viruses in different environments has been instrumental in abating or slowing the spread of different diseases, yet it clear that there is a need for faster, cheaper, and robust diagnostic tools that can be configured rapidly and can respond to emergent threats.

Embodiments of the present application relate to systems and methods for detection of viral particles, viral proteins, viral RNA or DNA. In particular, the systems and methods provided herein enable measurement of the presence and/or quantity of viruses, viral proteins, viral RNA or DNA in different fluids such as, but not limited to, blood, saliva, or viral transport media as well as any other fluid media of interest. Also multiplexed assays are put forward as well as parallel processing assays to detect qualitatively or quantitatively the presence of viruses, viral proteins, RNA, or DNA in multiple samples simultaneously.

### SUMMARY

Embodiments of the present invention relate to systems and methods for detection of viral particles, viral proteins, viral RNA or DNA, an area of special need where rapid and accurate diagnostic tools are necessary. In particular, the systems and methods provided herein enable measurement of the presence and/or quantity of viruses, viral proteins, viral RNA or DNA in different fluids such as, but not limited to, blood, saliva, or viral transport media as well as any other fluid media of interest. Also multiplexed assays are put forward as well as parallel processing assays to detect qualitatively or quantitatively the presence of viruses, viral proteins, RNA, or DNA in multiple samples simultaneously.

In the embodiments the methods consists of (i) functionalizing the surface of magnetic beads with a first composition stemming from molecules that can be antibodies, proteins or nucleic acids, and functionalizing a substrate with another composition, (ii) bringing the beads in contact with the substrate in a fluid that can (or not) contain viruses or viral lysates where antigens or viral RNA or DNA may be found. (iii) apply a magnetic torque to the beads so that they rotate and measure the rolling velocity of the beads using optical microscopy; and (iv) compare the rolling velocity to control samples to evaluate quantitatively or qualitatively the presence of viruses (virions) or molecules contained within them such as antigens and nucleic acids.

In another embodiment, the present invention is used to measure the presence of multiple kinds of viruses or antigens at the same time, a process called multiplexing. In another embodiment the system is configured to mimic natural viruses by constructing nanoscale objects that can concentrate many antigens on their surface. Such constructs are multivalent in nature and bind more strongly to the particles than single antigens, which is exploited to increase the signal.

Herein it is also put forward an apparatus that can perform the detection of viruses and associated proteins that can be completely automated and needs minimal user intervention, or conversely the user can perform all the steps separately. The apparatus incorporates a frame, coils for creating the magnetic fields, electronics for controlling the fields and running the currents, a touchscreen to control the apparatus and report the status of the sample, and a complete optical setup to capture the images from the samples in fluorescent or bright mode microscopy. The data is analyzed internally and reported. More details of the apparatus and other aspects and embodiments of the disclosed methods are described more fully in the detailed description.

### BRIEF DESCRIPTION OF THE DRAWINGS

FIGS. 1A and 1B are schematics of different modalities of systems using a rolling bead assay to detect quantitatively and semi-quantitatively: (A) Intact virions, (B) antigens, (c) RNA/DNA from viruses, and (d) RNA/DNA using proteins and selected fragments of RNA/DNA. In both A and B the functionalization is assumed to be protein based.

FIGS. 2A and 2B are schematics of different modalities of systems using a rolling bead assay to detect quantitatively and semi-quantitatively: (A) RNA/DNA from viruses, and (B) RNA/DNA using proteins and selected fragments of RNA/DNA.

FIGS. 3A and 3B are schematics of the different modalities of systems using a rolling bead assay to detect quantitatively and semi-quantitatively: (A) Intact virions, and (B) antigens using selected fragments of RNA/DNA. Sometimes these fragments are called aptamers.

FIG. 4 is a plot of the translational velocity of multiple beads as a function of time.

FIG. 5. Is a sketch of the core of the apparatus illustrating the different parts: the magnetic coils, the optics module which includes an illumination source, sample holder, optical lenses and detector(s), electronics for controlling the current in the coils and run the microprocessor that captures and processes the images as well as controls the overall apparatus, and finally a touch screen for interfacing with the operator. The system can have different coil configurations, here we show a 4 coil and a 6-coil configuration. The 6-coil configuration can create any kind of 3D magnetic field. The apparatus can analyze a single sample or multiple samples at the same time. Some configurations of the sensing module are displayed.

FIG. 6. Shows average normalized velocities of translation (also referred to as rolling parameter) for different assays as indicated in the figure. All samples are 100 uL in total and the number of nanoparticles is indicated in the figure.

FIG. 7 is a schematic of a multiplexed assay targeting two or more different intact viruses. The beads can be identified via their physical size or by labeling them with different fluorescent probes.

FIG. 8 is a schematic of a multiplexed assay targeting two or more different antigens or RNA/DNA or mixtures of thereof. The beads can be identified via their physical size or by labeling them with different fluorescent probes.

FIG. 9 is a schematic of the rolling bead assay using a multivalent construct where the analyte of interest is preconcentrated on the surface of a nanoparticle or other chemical construct to bind multivalently with the bead and the substrate. These multivalent constructs mimic full virions.

FIG. 10 is a plot of the normalized velocity of a streptavidin coated bead rolling on a biotin functionalized substrate as a function of the concentration of added avidin, a blocking protein. The number of active binding sites can be calculated from the equilibrium constant for this system which is very low, of the order of 10⁻¹⁵.

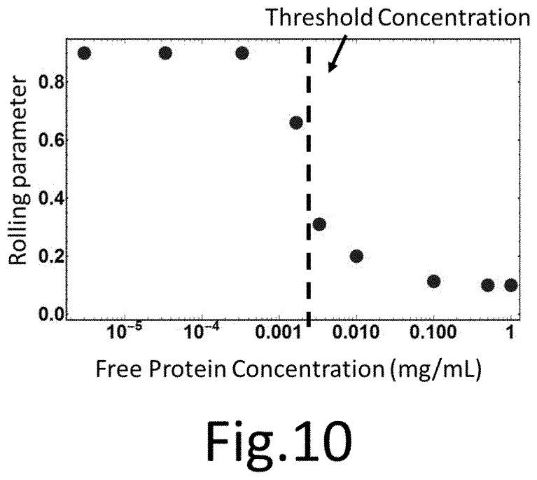

### DETAILED DESCRIPTION OF THE INVENTION

A simple, fast, and robust method is provided for measuring the presence and quantity of multiple different kinds of viral particles (virions), viral proteins, RNA, or DNA in different fluids such as, but not limited to, saliva, blood or mucus from a nasopharyngeal swab. Embodiments described herein address the foregoing needs to providing rapid, sensitive, and cheap diagnostics systems for different analytes. The system can detect intact viral particles (virions), or viral proteins, DNA, or RNA in a qualitative and quantitative fashion.

### Method and Apparatus

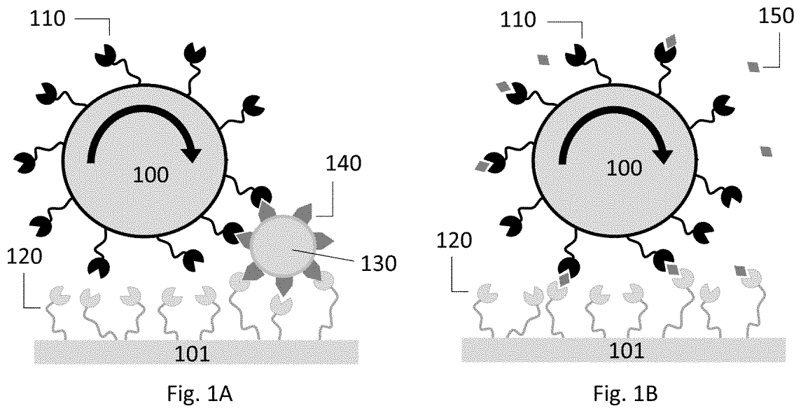

The present invention relates to detecting the presence of a virus, viral protein, RNA, or DNA in different fluids. The fluids can be for example, but not limited to, saliva, blood, serum, viral transport media, saline buffer, extraction buffers, or other liquids of interest including mixtures of the aforementioned. The method herein put forward consists of functionalizing the surface of magnetic beads with one or more types of molecules such as antibodies, proteins, or nucleic acids (RNA or DNA). Synthetic or organic molecules that bind to the motif of interest can also be used. The functionalization can be done via a secondary binding event using for example biotin-streptavidin linkages. A substrate has to be also functionalized with molecules (antibodies, nanobodies, proteins, nucleic acids, aptamers) that can be of the same species as those in the beads or different. The beads are deposited in a fluid and are allowed to come into contact with the substrate. Finally, the beads are set in motion by applying a magnetic field that rotates in a particular plane and at a particular frequency. The presence of viruses, viral proteins, RNA, or DNA in different fluids can then be obtained by measuring the translational velocity of the beads. FIGS. 1A and B, 2A and B and 3A and B illustrates this process in different modalities. In particular one can detect the presence of intact viruses or proteins antigens by functionalizing a magnetic bead 100 with antibodies or proteins 110. The bead rotates as indicated in the arrow due to a magnetic torque. When the bead is near a substrate 101 functionalized with the same protein or antibody or a different complementary kind 120, the bead will change it translational velocity depending if there is a mechanical linkage due to the presence of 1 or more virus(es) 130 bound to both the bead and the substrate via proteins on their surface 140, as shown in FIG. 1A. The linkage can be also constructed using viral antigens 150 as shown in FIG. 1B. Once this linkage(s) are stablished, the friction with the substrate increases providing more thrust to the bead and thus an increase on the translational velocity of the beads.

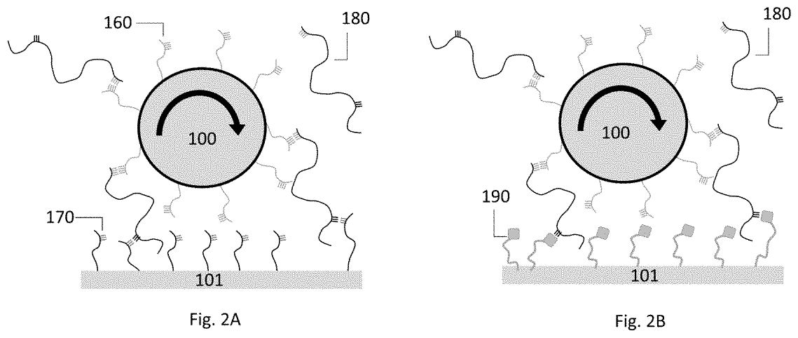

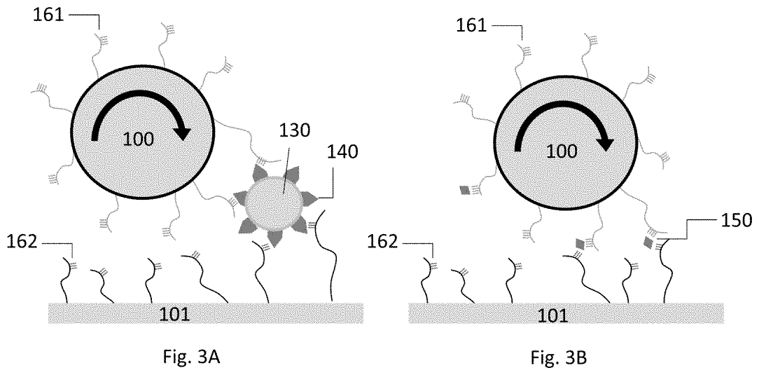

The same principles apply if instead of functionalizing the beads with proteins or antibodies one uses nucleic acid constructs such as DNA or RNA stands 160. In order to create a link, another DNA or RNA functionalizing motif has to be added to the surface 170. Using these constructs one can detect complementary bonding of DNA or RNA fragments 180 coming from the viruses as shown in FIG. 2A. Notwithstanding, the substrate or the bead or both can be functionalized with a RNA or DNA binding protein 190 as shown in FIG. 2B. Another option is to use nucleic acids that bind particular viral proteins on both the beads 161 as well as in the substrate 162 to detect full viruses or viral antigens as shown in FIGS. 3A and B. Clearly, a combination of nucleic acids with proteins or antibodies can also be used.

The critical quantity that one measures in the present invention is the translational velocity of the rotating beads. Such quantity can be extracted from video microscopy taken from the sample while the rotating magnetic field is actuated. Once the translational velocity is obtained one can then compare this velocity to a control sample and determine the presence, or not, of intact viruses, antibodies, viral proteins, RNA, or DNA. FIG. 2 exhibits a typical set of trajectories of the beads as obtained from the microscopy set. To obtain the translational velocity one uses a tracking algorithm which is done on a computer or microprocessor. There are several commercial or open-source tracking algorithms available at the present time. In the examples that will be presented within the present invention, a proprietary code is utilized to perform the tracking and analysis of the translational velocities of the beads.

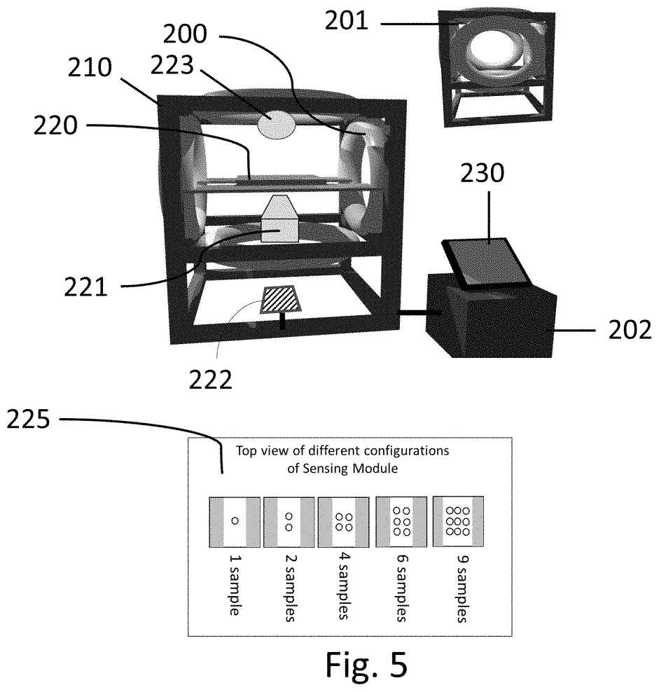

FIG. 5 shows a diagram of the apparatus where the results presented herein were obtained. To generate the magnetic fields one can use different wave generators and different coil set-ups. The signal is then amplified using commercial or custom-made amplifiers. Also, current microcontrollers such as Arduinos are equipped to generate such waves. The most widely use setup are Helmholtz coils, since they provide the most homogeneous fields. Equal distance separated coils. or X-configured coils all work for the present invention. In FIG. 5 we show a 4 coil configuration 200, as well as a 6 coil configuration 201 with complete control of the 3D magnetic field. The key step to make rotating magnetic fields is to run currents in the coils that are in different orthogonal axes with a phase lag of 90 degrees. The different sine or cosine signals can be generated with a computer and sent to the coils via amplifiers. In the present invention the computer(s), microcontrollers, power supplies and amplifiers are all housed in an external box 202. Not withstanding, the components can also be housed within the frame of the apparatus 210 making everything self-contained in a single apparatus. The frequency range for the rotation of the coils ranges from 0.01 hz to 100 hz. Amplifiers capable of working in this frequency range must be used. The signal from the computer or integrated circuit may need to be converted to an analog signal. This is done using digital-analog-converters (DACs). Once the system is integrated, the torque on the beads is proportional to the magnetic moment of the beads (or bead chains) and the strength of the magnetic field. The torque is then written as

torque=μB

Where B is the magnetic field strength and μ is the magnetic moment. The magnetic moment depends on the particular bead one is using. In the case of superparamagnetic beads the magnetic moment is proportional to the field. In the case of ferromagnetic beads, the moment is fixed once the particles are magnetized. The magnitude of the magnetic field is controlled by the current flowing through the coils and the number of turns per coil. Here we envision systems that can have a minimum of 10 windings per coil to 10000 windings per coil. The typical magnetic field necessary for the method here presented can vary from 0.01 mT to 100 mT, where T refers to the magnetic field scale of Tesla. The shape of the coil can be circular or rectangular.

The imaging of the sample is accomplished by using a sample holder 220, a lens or set of lenses 221 to amplify the images from the samples and a sensor or sensors 222 are used to capture such images and make them in digital format amenable for the microprocessors or computers to analyze as explained before. The sample(s) are illuminated using a light source 223. The apparatus can be used in different modalities such as bright field microscopy or fluorescence microscopy for which one would need to add more filters in the optical path.

The system here put forward is automated. The user only interacts with a touch screen 230 which directs the user to perform different tasks and run the sample as indicated. In particular, the screen guides the user to do each of the steps in sample preparation, sample viewing, and finally sample analysis. The system can also be run manually where the user does the steps independently and can at any time request any of the functions such as viewing, looking for instructions, or simply bypass all of this and run the sample.

The system can also run multiple samples in parallel using different configurations for the sample formats 225, as well as using a single or multiple lenses 221 for amplification, and a single or multiple cameras as sensors 222 which can be used to measure different wells, channels, or other sections of the same sample. The present invention is compatible with industry used formats such as 96/384 single, strip or full plates wells, microfluidic samples on transparent substrates, as well as commonly used glass slides and microwells. The results from each of the different samples can be provided at the end. FIG. 5 shows a diagram of the apparatus and a photograph of a functioning system where the results for the results presented herein.

### Magnetic Beads

The magnetic beads 100 can be of different kinds including ferromagnetic, paramagnetic, or superparamagnetic. The bead material has to be such that one can functionalize it surface either by adsorption, by chemical modification, or by direct interactions (e.g. biotin-streptavidin, avi-tag, his-tag, etc). Many of these beads are currently available in the market. The size of the beads required are only limited by being able to be observed in a microscope. Thus, any bead that is larger than 250 nm and smaller than 1000 μm can be used in the present invention.

### Substrates

The substrate has to be flat and one needs to be able to functionalize it either by adsorption, by chemical modification (e.g. covalent chemistries), or by direct interactions (e.g. biotin-streptavidin, avi-tag, his-tag, etc). It is possible to use slides, coverslips, or wells with flat bottoms. 96-well plates and 384-well plates can also be used. Other substrates that are transparent and allow for microscopical observation can also be used. Microfluidic devices that have the properties mentioned above can also be used. For example, slides with microfluidic channels are a way to increase throughput from a single slide. Other microfluidic substrates are amenable as well as long as one can image the beads through the system.

### Multivalent Constructs

Multivalent constructs can be performed using nanoparticles, liposomes, or other nanocontructs of different sizes with different core chemistries and a functional surface. Many of these nanoparticle are available in the market. The size of the nanoparticles, liposomes or nano-constructs can range from 5 nm to 1 μm. Such nanoparticles, liposomes or nanoconstructs need to be functionalized with proteins on their surface to bind to a target antigen, RNA or DNA. The functional molecules on the surface can be for example, but not limited to, proteins, antibodies, DNA fragments. The composition of the nanoparticles can vary from plastic, polymer or metal, or a combination of the former. The surface can have many different compositions. The liposomes can be of many kinds of different lipids such as DOPC, DOPS, DOG or other lipid types. It can include cholesterol and functional lipids onto which one can functionalize the liposome.

### Samples

The sample that is going to be measured may have different concentrations of the analyte of interest as can be intact virions, antigens, etc. The fluids that one can work with comprise, but are not limited to saliva, blood, serum, buffer, extraction buffers, lysates, water or other solvents, transport media, mucus from nasopharyngeal samples, or lysates of the aforementioned media. The presented methods are useful in a concentration range from 100 viral copies/mL and above. Copies refers to the number of viral particles present in one mL of solution. The number of viral proteins depends on the protein type and the number of copies per virus each has. For example, in the case of SARS-COV-2 the most prominent antigen is the N protein of which there are on average 3000 copies per virus. For this protein, the limit of detection is thus of the order of 300,000 proteins per mL which is in the sub femtomolar range.

### Modalities of the System

### 1. Detection of a Single Virus-Type

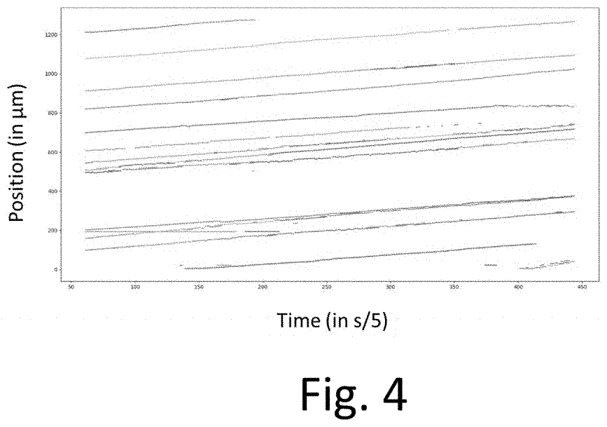

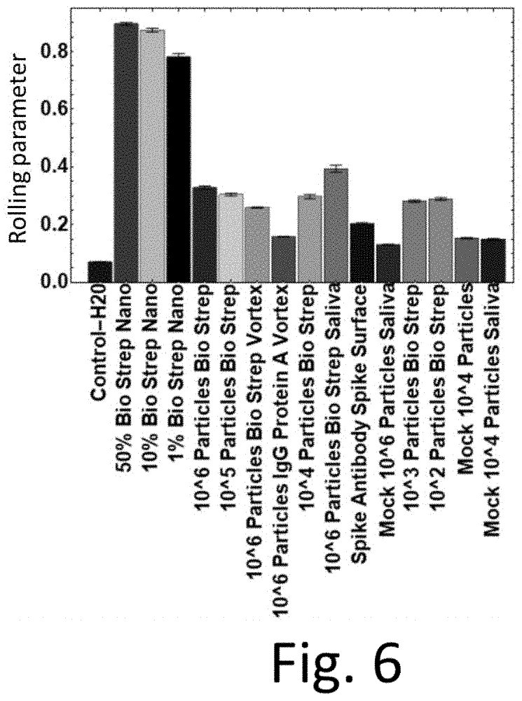

In the case one is interested in detecting a single virus one can use the present invention by functionalizing the magnetic beads with an antibody on their surface that binds to the virus. The substrate can be functionalized with the same antibody. Such antibodies are commercially available or can be generated in-house using standard protocols. It can be monoclonal or polyclonal antibodies. In the present example we use a polyclonal antibody from Abeomics to the SARS-CoV-2 Spike protein. Also, one can use multiple different proteins on the surface to functionalize the beads. Different combinations of proteins will bind in a differentiated manner different mutants of say a given virus. This might be beneficial in case one would like to discriminate two different variants of related viruses and make it very specific to one of them. In the case of Influenza, where there are several variants, for example, this could be important. Once the surfaces are functionalized, a solution containing the virus is mixed with the solution containing the magnetic beads. The mixing period can vary between 10s (basically just solution preparation time) and 24 hours. If left for long to mix it is recommended to do so between 2-8 degrees C. Typically one mixes for at least 1 min. The mixing protocol can be by vortexing, pipetting, or manual agitation. Also, one can leave the samples on the bench and wait for them to mix passively by diffusion. Once the solution is ready, it is placed on the substrate using a pipette. Under some conditions it might be convenient to re-concentrate the beads in case one uses small quantities in the mixed solution with the virus or antigens. To do so it is possible to use a centrifuge and take the final sample for sensing from the bottom of the well or tube. In this particular example, ferromagnetic beads are used and they can be magnetized with a permanent magnet before being placed in the magnetic field from the coils, but they also work if they are not magnetized before. If the beads are paramagnetic or superparamagnetic there is no need for a premagnetization step. Once the sample is ready with the solution containing magnetic beads and viral particles on the substrate, one can start to rotate them at a frequency between 0.01 hz and 100 hz. Typical values are 0.1 hz to 10 hz. The bead motion is recorded or obtained on-the-fly and the translational velocity of the beads is obtained. A simple dimensionless number that can be directly compared across beads is the normalized velocity (or rolling parameter) defined as

$$\overline{ζ} = \frac{v}{2 π fR}$$

Where v is the velocity, f the frequency of rotation, and R is the radius of the bead. This quantity varies between 0 and 1. A value of 0 means the bead does not translate and there is no interaction with the substrate whatsoever. It is rare to find this number except for stuck beads since one always has some hydrodynamic friction. A value of 1 is perfect rolling. Once this quantity is obtained one can directly compare to a sample without virus to know if a virus is present or not and also see what the limit of detection is. In FIG. 4 we show traces of beads (Spherotech Ferromagnetic of 10 μm in size) functionalized with anti-SARS-CoV-2 antibodies (Abeomics Inc.) in positive samples of Covid-19 (IBT, UNAM). In FIG. 6 we show different average normalized velocities (i.e. rolling parameters) as a function of concentration of model viral particles per mL. As can be seen, for strong interactions the limit of detection is below 10³ viruses/mL, comparable to some RT-PCR machines. The time to detection is rather quick and can be done in less than 1 minute. The overall time in the figure used was 80 seconds. The limit of detection will depend on the particular strength of the interactions between the viral surface and the proteins, RNA, or DNA used to functionalize the beads. In the case of antibodies, their affinities are typically in the range of nano Molar (nM), and given that a virus has many proteins on its surface one expects to see overall affinities in the pico Molar (pM) to femto Molar (fM), implying that the viruses will stay stuck on the surface of the beads. Binding through many different proteins is called multivalent binding and it is prevalent in biology. The virus, however, may rotate since this does not incur in large energy penalties, and corresponds to breaking a single bond at a time. In the particular system we have studied with a viral particle of 150 nm that mimics SARS-CoV-2 the limit of detection is less than 1 virus per particle. In FIG. 6 we present the different results for the rolling parameter for different interaction strengths between viruses and beads. We find that the limit of detection of this system can go as low as 100 copies/mL. This detection limit is comparable to molecular diagnostics, which is much more sensitive than antigen tests.

The test can be applied to different kinds of viruses such as Sars-Cov-1, Sars-Cov-2, Influenza A, Influenza B, Influenza C, Influenza D, Zika, Ebola, Denge, Rotaviruses, Coronaviruses, RSV virus, Herpes, HIV, Epstein-Barr Virus, Hepatitis viruses, etc. This list is meant as an example, but not limiting.

### 2. Multiple Virus Types

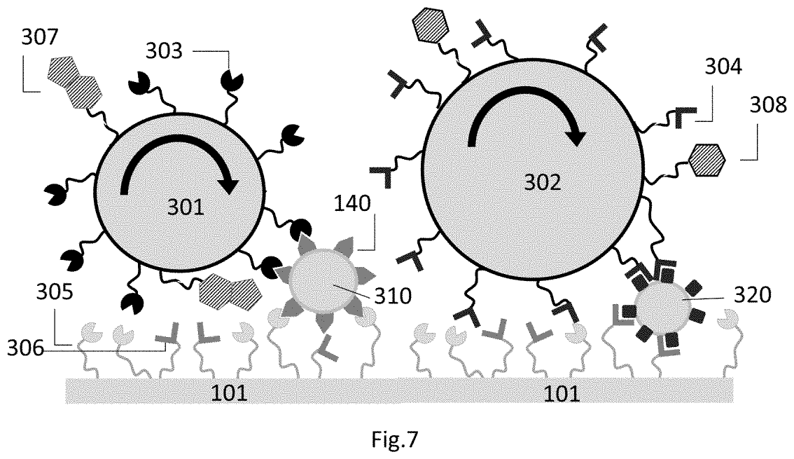

In order to be able to detect multiple different viruses at the same time, the present invention can be used as well. The process of being able to detect multiple different species is called multiplexing. In this particular case, multiplexing is done on the same sample. To do this in the current method one needs to have different types of beads with different proteins on their surfaces. A particularly useful construct is using antibodies since they have high affinity to particular proteins in the surface of the virus. To distinguish the different beads one can use either fluorescent tags with different colors or simply use beads of different size. In a typical sample one should be able to detect between 1-10 different viruses. In FIG. 7 we show how viral multiplexing is done using 2 different types of beads 301 and 302, which are functionalized with different molecules (which can be proteins, antibodies, nanobodies, aptamers, DNA or RNA). On bead type 1 301 one can use one type of functionalization 303 and in bead type 2 302 one can use a different functional group 304. On the substrate one can functionalize with two por more types of molecules 305, 306 from the molecular repertoire mentioned above for the beads. These molecules can be the same or different to those in the beads. However, both the molecules functionalizing the beads and the substrate have to bind in a differentiated way to two different types of viruses. For example, the functional groups in bead type 1 as well as one of the functional groups in the substrate bind to a virus of type 1 310, and consequently the functional groups in bead type 2 and the substrate should also be able to bind to virus type 2 320. To identify the different beads it is possible to use size of the beads or different fluorescent tags for each bead. For example, bead type 1 can be tagged using a fluorescent tag 307 and bead type 2 can be tagged with a different fluorescent tag 308.

### 3. Viral Antigens: Proteins, RNA or DNA

While one can detect intact viruses using the present invention, one can also detect viral proteins, RNA, or DNA. This is done first by lysing the solution and then detecting the antigens, RNA, or DNA directly by using either antibodies, other proteins, or complementary nucleic acid strands to functionalize the surface of the beads. Such molecules on the surface of the beads will capture the antigen (i.e. viral protein) or the nucleic acid material while mixed in a solution that has the viral proteins or RNA or DNA. Examples of molecules useful for the functionalization are: antibodies to a particular protein, proteins that bind specific sequences of nucleic acids, and small RNAs or DNAs that will specifically bind a target sequence (e.g. primers for PCR or aptamers). As in the previous modalities, one also needs a functionalized substrate on which to roll. The substrate can be functionalized with a complementary protein (or antibody), complementary RNA or DNA strand. The idea is that once the bead has captured an antigen, when it brings it close to the substrate both will sandwich the antigen and create a bond between the magnetic bead and the surface. This will increase the velocity of translation and thus one will detect the virus via the presence of viral antigens.

In a particular example, one might be interested in targeting different regions of a viral RNA. The substrate and the beads can be functionalized with different short sequences of complementary RNA to bind to those regions. A schematic of some of the different molecular bonds that can be formed is shown in FIGS. 2A and 2B.

### 4. Multiple Different Viral Antigens or Proteins

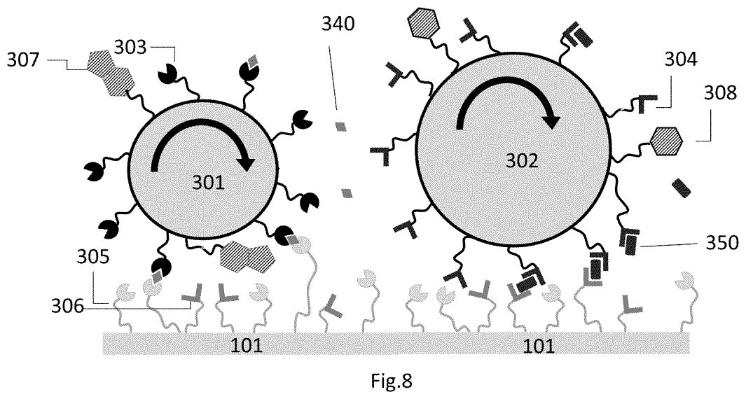

As in the case of multiple intact viruses, one can also detect multiple different viral strands by multiplexing. An example of this variant of the technique is shown in FIG. 8. Using different functionalizations on different beads and in the substrate as was shown previously for virions in FIGS. 7A and B, it is possible to detect 2 or more antigens 340, 350 coming from different kinds of viruses. Another interesting alternative is to detect multiple antigens from the same virus. In this case, however, the functionalizing molecules have to be directed to bind to the antigens of interest instead of to the surface of the viruses.

### 5. Multivalent Constructs for Enhanced Antigen Detection Using Bead Rolling as a Sensor

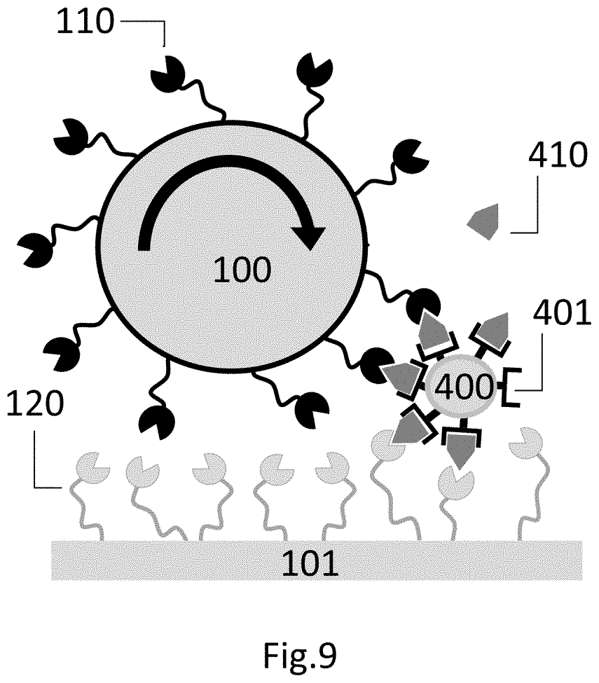

If one is interested in mimicking the effect of intact viruses on the motion of the rolling beads, and thus exploiting multivalency, it is possible to include an intermediate multivalent construct that mimics a virus as shown in FIG. 9 400. In this case one uses nanoparticles, liposomes, or similar objects that can move rapidly and easily through fluids to capture antigens 410, and in particular viral proteins. In order to capture specifically a given antigen, it is necessary to functionalize the surface of the multivalent constructs with molecules 401 such as, but not limited to antibodies, proteins or nucleic acids that can bind selectively such antigens. The nanoscale multivalent constructs can then be captured by the magnetic beads 100 through multivalent binding, just as is the case for an intact virus (see FIG. 1B). When the magnetic particles roll on the surface their translational speed will increase due to the binding to the substrate mediated by the multivalent constructs. Such constructs can be useful in situations where the binding is weak, and multivalency renders them strong binders. Results from model (mock) viruses were generated this way using a biotin streptavidin linkage to bind viral antigens to a nanoparticles and render it multivalent. Results shown in FIG. 5 are from such constructs.

### 6 Quantitative Detection of Protein Concentration in a Single Sample

By forming a gradient of binding in a sample, which can be performed in multiple ways from simply removing a substrate from a solution used to functionalize at a specific rate, to more sophisticated methods using microprinting technology. Once a gradient is formed, it is possible to find the density of binding sites by comparison to known densities in a homogenous substrate. This is the calibration step and can be done apriori. However, once a gradient in binding sites in the substrate has been formed it is possible to use this substrate to obtain the concentration of an analyte, as could be a viral antigen, in solution. This is done by finding the concentration range on the surface at which the bead translational velocity drops from the saturated regime to the unsaturated regime. It can be done in a competitive assay or in sandwich assay. The length of the sample over which the gradient is formed can be between 100 μm to 1 cm. Another modality can be to use a mixture of different beads tagged fluorescently as is done for multiplexed assay described before in which each bead has a different concentration of functional groups in their surface. In this case it is not necessary to form gradients.

The equilibrium concentration of an analyte, it being a virus or an antigen, is determined then by the binding affinity of the analyte to the substrate and the concentration in solution of the analyte. However, if the binding motif in the substrate gets diluted, the binding rate per motif remains constant at a given concentration, but the overall concentration of analyte adsorbed in the surface is not constant. It is proportional to the surface coverage. In ELISA tests, this is the so-called linear regime when different titrations are performed. In the present invention one can do this directly in a single well, or single sample by using the gradients or different beads with different concentrations of functional groups on their surface. The translational velocity as a function of the concentration on the surface will be different. Once the substrate is placed in a solution containing the analyte at a given concentration one will have a gradient in bound analyte in the surface. However, the offset will be different for different concentrations. Also, the rolling velocity is logarithmic on the affinity, so it is a very sensitive measure of different affinities. As the beads roll on the substrate one can find where the beads transition from a state of mostly roll without slip to a state of slipping. In terms of the rolling parameter one expects a drop from a rolling parameter found at saturation to one which would be the baseline for a sandwich assay. Conversely, if one uses a competitive assay, one expects to see the rolling parameter increase for a particular region in the substrate. This region indicates the concentration of analyte present in solution. It will be different for each different analyte and concentration and must be calibrated apriori. An example of this behavior is shown in FIG. 9 for a magnetic bead functionalized with biotin rolling on an avidin substrate. The solution contains free avidin. As the concentration of free avidin increases one sees a precipitous drop in the rolling parameter. Conversely, one can think of this as a substrate with less binding sites as the concentration is increased.

1. A method to detect the presence of viral particles in different fluids comprising:
Magnetic bead(s) with a surface functionalization of first composition in contact with a substrate functionalized with a second composition in a solution containing intact virions, viral antigens RNA or DNA. The first composition may be the same as the second composition.
Applying a magnetic torque to rotate the beads and make them move across the substrate
Measuring the velocity of translation of the rolling magnetic beads
Determining if there is virus or viral antigens, RNA or DNA present in a qualitative (positive or negative), semi-quantitative or quantitative fashion.

2. The method of claim 1 where the detection of the motion of the beads is done using optical microscopy.
The method of claim 1 where the detection is of a single sample as described herein.
The method of claim 1 where the detection is of 2 samples simultaneously as described herein.
The method of claim 1 where the detection is of 4 samples simultaneously as described herein.
The method of claim 1 where the detection is of 6 samples simultaneously as described herein.
The method of claim 1 where the detection is of 8 samples simultaneously as described herein.
The method of claim 1 where the detection is of 9 samples simultaneously as described herein.
The method of claim 1 where the detection is of 16 samples simultaneously as described herein.
The method of claim 1 where the detection is of 96 samples simultaneously as described herein.
The method of claim 1 where the detection is of 384 samples simultaneously as described herein.

3. The method of claim 1 where one uses two or more types of beads each having a particular composition that is different between the different types of beads as described herein, and a substrate that has 1 or more different compositions.

4. The method of claim 1 where one can detect 1 or more different viruses at the same time using multiple compositions for the magnetic beads and substrates as in claim 3 and described herein.

5. The method of claim 1 where one can detect intact virions as described herein.

6. The method of claim 1 where one can detect viral antigens as described herein.

7. The method of claim 1 where one can detect viral RNA or DNA as described herein.

8. The method of claim 1 where one can detect 1 or more different kinds of intact virions in the same sample as described herein.

9. The method of claim 1 where one can detect 1 or more different kinds of viral antigens in the same sample as described herein.

10. The method of claim 1 where one can detect 1 or more different kinds of viral RNA or DNA in the same sample as described herein.

11. The method of claim 1 where one uses a 2 axis magnetic field generator with 4 coils

12. The method of claim 1 where one uses a 3 axis magnetic field generator with 6 coils

13. The method of claim 1 where one can quantitatively evaluate the concentration of viruses by using a gradient in the second composition (the substrate composition) as described herein.

14. The method of claim 1 where one can quantitatively evaluate the concentration of viral antigens employing a gradient in the second composition (the substrate composition) as described herein.

15. The method of claim 1 where one can quantitatively evaluate the concentration of viral RNA or DNA employing a gradient in the second composition (the substrate composition) as described herein.

16. The method of claim 1 where the first composition can be proteins in the following families: antibodies, antigens, and receptors.

17. The method of claim 1 where the first composition can be nucleic acid sequences between 5 and 2000 base pairs in length.

18. The method of claim 1 where the second composition can be proteins in the following families: antibodies, antigens, or receptors, and can be the same or different from the first composition.

19. The method of claim 1 where the second composition can be nucleic acid sequences between 5 and 2000 base pairs in length, and can be the same or different from the first composition.

20. The method of claim 1 where the first composition can be a 1 or more different proteins from the following families: antibodies, antigens, and receptors.

21. The method of claim 1 where the magnetic beads can be ferromagnetic, paramagnetic or superparamagnetic.

22. The method of claim 1 where the magnetic beads can have a diameter between 250 nm and 1000 μm.

23. The method of claim 1 where the substrate can be part of a slide, a coverslip, a well in a 96-well plate, or a well in 384-well plate.

24. The method of claim 1 where the substrate can be a microfluidic channel.

25. The method of claim 1 where the substrate has lateral dimensions above 100 μm and below 10 cm.

26. A system to detect the presence of viruses using the method of claim 1 that
Utilizes a rotating magnetic field produced by magnetic coils as described herein.
The translational motion of the beads is captured by optical microscopy or fluorescence microscopy as described herein.
A microprocessor is utilized to track the motion of the beads and obtain the translational velocity as described herein.
A microprocessor is utilized to provide a result on a touch screen or in a computer screen as described herein.
The system can run in automatic mode, be coupled to a robot, or be manually controlled as described herein.

27. The system of claim 26 where the user controls the operation of the system using a touch screen as described herein.

28. The system of claim 26 where the user controls the operation of the system using a computer as described herein.

29. The system of claim 26 where the user controls the operation of the system using a computer.

30. The system of claim 26 where the sensing module can process 1 sample at a time as described herein.

31. The system of claim 26 where the sensing module can process between 2 and 384 samples at the same time using 1 or more lenses and a large camera sensor or multiple smaller sensors as described herein.

32. The method of claim 1 where one can quantitatively evaluate the concentration of viral antigens employing multiple concentrations of the functionalization of the first composition (the bead composition) as described herein.

33. The method of claim 1 where one can quantitatively evaluate the concentration of virions employing multiple concentrations of the functionalization of the first composition (the bead composition) as described herein.

34. The method of claim 1 where one can quantitatively evaluate the concentration of viral DNA or RNA employing multiple concentrations of the functionalization of the first composition (the bead composition) as described herein.
